(19) United States
(12) Patent Application Publication
(10) Pub. No.: US 2024/0226530 A9
(43) Pub. Date: Jul. 11, 2024

(54) RESEALING CAP FOR INTRAVENOUS (IV) BAG SPIKE PORT

(71) Applicant: Nicole E. HINCKLE, Souderton, PA (US)

(72) Inventor: Nicole E. HINCKLE, Souderton, PA (US)

(21) Appl. No.: 18/049,539

(22) Filed: Oct. 25, 2022

(51) Int. Cl. A61M 39/20

(52) U.S. Cl. CPC A61M 39/20

(57) ABSTRACT

A resealing cap for an intravenous (IV) bag spike port is shown and described. The resealing cap for an intravenous (IV) bag spike port includes a housing having a first end and a second end. the housing has a passageway therethrough. The first end of the housing is a closed end. The closure is created by a puncturable material. The second end of the housing is an open end.

### BACKGROUND OF THE INVENTION

The present invention relates to medical devices and training. More particularly, the present invention provides a resealing cap for an intravenous (IV) bag spike port.

Currently, IV bags are regularly used in the medical field. These bags are typically disposable. However, in spite of the large volume of use and the disposability of IV bags, they can still be extremely costly. Currently, an IV bag may only be used once. The reason for this is due to the spike port of the bag. Spike ports are sealed with a membrane, which is punctured in order for the interior liquid of the bag to enter IV tubes. Once punctured the membrane is destroyed and this the bag cannot be reused; even for training or other purposes where a bag could be reused.

This lack of reusability means that many bags are used throughout the course of training. In a training setting bags may be spiked and then discarded without ever administering any fluids at all. This means that the cost to train someone may be astronomically high. Or on the other hand a person's training may be limited due to the cost.

in a second example, only a partial IV bag may be required to administer a dose of medication. In these instances, the bag, without a way to properly reseal it, will be thrown away and a new bag would be needed for the next dose. This leads to a higher cost for treatments where an IV bag is required.

Consequently, there is a need for an improvement in the art of medical training and recusing supplies. Further improvement is needed in the art of resealing IV bags and spike ports. The present invention substantially diverges in design elements from the known art while at the same time solves a problem many people face when training using IV bags. In this regard the present invention substantially fulfills these needs.

### SUMMARY OF THE INVENTION

The present invention provides a resealing cap for an intravenous (IV) bag spike port wherein the same can be utilized for providing convenience for the user when using a resealing cap for an intravenous (IV) bag spike port. The resealing cap for an intravenous (IV) bag spike port includes a housing having a first end and a second end. the housing has a passageway therethrough. The first end of the housing is a closed end. The closure is created by a puncturable material. The second end of the housing is an open end.

Another object of the resealing cap for an intravenous (IV) bag spike port is to have the housing be made from a medical grade material.

Another object of the resealing cap for an intravenous (IV) bag spike port is to have the housing be made from an elastic material.

Another object of the resealing cap for an intravenous (IV) bag spike port is to have the second end of the housing has a removable seal placed about the open end.

Another object of the resealing cap for an intravenous (IV) bag spike port is to have the housing be cylindrical.

Another object of the resealing cap for an intravenous (IV) bag spike port is to have a plurality of threads located around the interior of the housing.

Another object of the resealing cap for an intravenous (IV) bag spike port is to have knurling about the exterior of the housing.

Another object of the resealing cap for an intravenous (IV) bag spike port is to have a pull tab located at the second end of the resealing cap.

Another object of the resealing cap for an intravenous (IV) bag spike port is to have an extension tube located between the first end and the second end of the resealing cap.

Another object of the resealing cap for an intravenous (IV) bag spike port is to have a tab protruding from the resealing cap.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of a resealing cap fitted to an IV bag.

FIG. 2 shows a perspective view of an embodiment of the resealing cap fitted to an IV bag with a threaded spike port.

FIG. 3 shows a perspective view of an embodiment of the resealing cap with threads fitted to an IV bag with a threaded spike port.

FIG. 4A shows a bottom-up view of an embodiment of the resealing cap.

FIG. 4B shows a cross-sectional view of an embodiment of the resealing cap.

FIG. 5 shows a perspective view of an embodiment of the resealing cap.

### LIST OF REFERENCE NUMERALS

With regard to the reference numerals used, the following numbering is used throughout the drawings.

| | |
|---|---|
| 100 | Resealing cap |
| 101 | IV bag |
| 102 | Exit port |
| 103 | Entracne port |
| 104 | Spike |
| 105 | Line |
| 106 | Needle |
| 107 | Threads |
| 110 | Housing |
| 110a | First end |
| 110b | Second end |
| 110c | Interior |
| 111 | Threads |
| 112 | Knurling |
| 113 | Sealing material |
| 501 | Extention tube |
| 502 | Tab |
| 503 | Pull tab |

### DETAILED DESCRIPTION OF THE INVENTION

Reference is made herein to the attached drawings. Like reference numerals are used throughout the drawings to depict like or similar elements of the resealing cap for an intravenous (IV) bag spike port. For the purposes of presenting a brief and clear description of the present invention, a preferred embodiment will be discussed as used for the resealing cap for an intravenous (IV) bag spike port. The figures are intended for representative purposes only and should not be considered to be limiting in any respect.

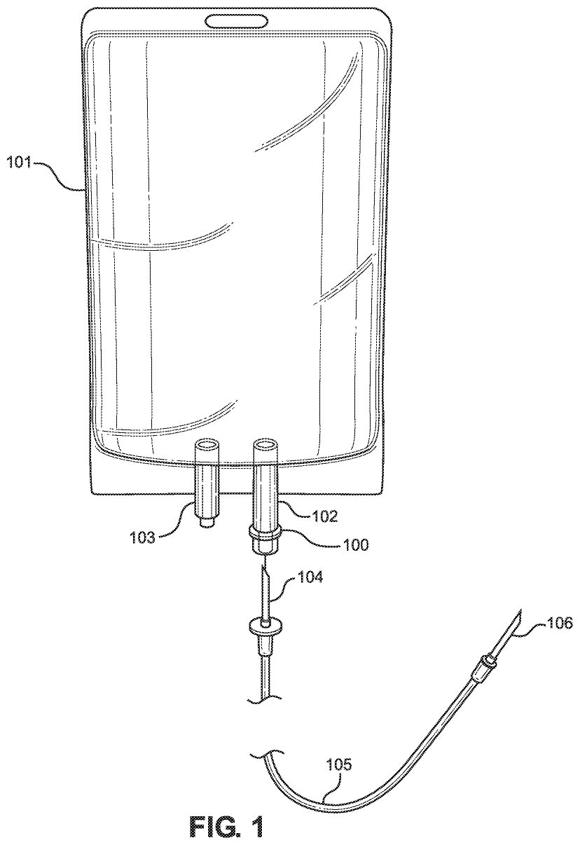

Referring now to FIG. 1, there is shown a perspective view of an embodiment of a resealing cap fitted to an IV bag. IV bags 101 and other such items are regularly used in the medical industry. Typically, these items have at least one exit port 102. In some embodiments the IV bags also include at least one entrance port 103. Typically, in order to access the exit port 102 a spike 104, is used to puncture a membrane which seals the exit port 102. The spike 104 is connected to at least one line 105 which leads to a needle 106 for injection of the substance in the IV bag 101. Currently, once the spike 104 is used to puncture the membrane of the exit port 102 the IV bag 101 cannot be resealed.

In some cases, proper resealing may be desired. In the shown embodiment a resealing cap for an intravenous (IV) bag spike port 100 is placed about the exit port 102 of the IV bag 101. The physical makeup of the resealing cap 100 will be described in detail below. In one embodiment the resealing cap 100 is of the size necessary to fit over the exit port 102. On another embodiment the resealing cap 100 is made of at least one elastic material. This will allow the resealing cap 100 to expand and contract as the resealing cap 100 is placed about the exit port 101.

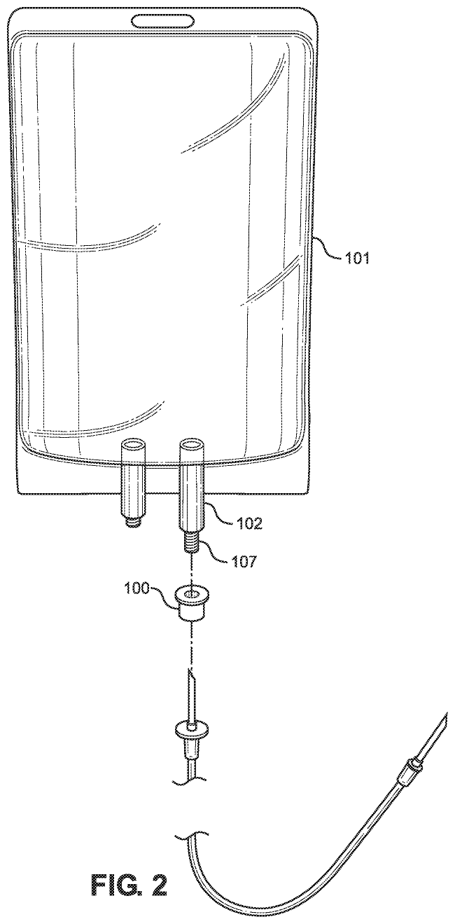

Referring now to FIG. 2, there is shown a perspective view of an embodiment of the resealing cap fitted to an IV bag with a threaded spike port. In some cases the exit port 102 of an IV bag 101 includes threads 107. In one embodiment the as previously mentioned the resealing cap 100 is made from at least one elastic material. In this embodiment the elasticity of the resealing cap 100 will allow for the resealing cap to be fitted about the threads 107 of the exit port 102. In this way the resealing cap 100 will be fitted to the exit port 102 of the IV bag 101.

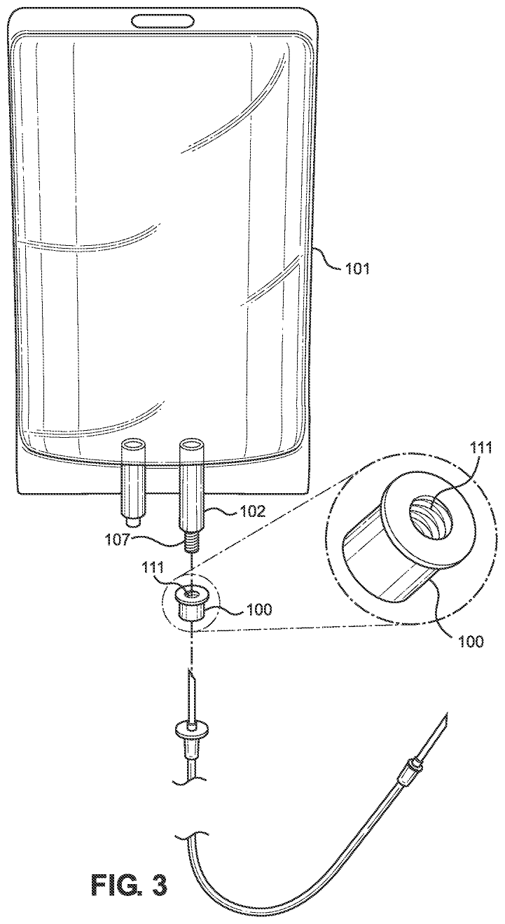

Referring now to FIG. 3, there is shown a perspective view of an embodiment of the resealing cap with threads fitted to an IV bag with a threaded spike port. In another embodiment the resealing cap 100 includes a plurality of threads 111. The plurality of threads 111 will also for the resealing cap to be secured to the threads 107 of the exit port 102 of the IV bag 101. A thread-to-thread connection has the benefit of being an easily secured. It is possible that a friction fit connection will be more difficult to properly connect. This could be a further drawback in a situation where speed is required.

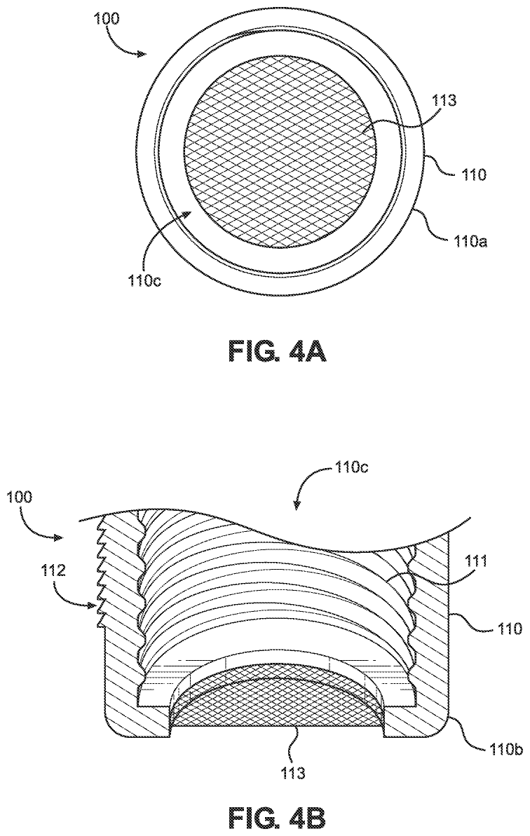

Referring now to FIG. 4A and FIG. 4B, there is shown a several views of an embodiment of the resealing cap. The resealing cap 100 includes a housing 110. The housing 110 has a first end 110a and a second end 110b. In one embodiment the housing 110 is cylindrical. In one embodiment the housing 110 is made from at least one elastic material. In another embodiment the housing 110 is made from a rigid material. In one embodiment the exterior of the housing has a knurling 112 thereon. This will allow for a user to ensure they have a good grip for installing and removing the resealing cap 100.

As mentioned about in one embodiment the interior 110c of the resealing cap 100 has a plurality of threads 111. The threads will allow for the resealing cap to be quickly installed on an exit port of an IV bag. The threads will allow for the exit port to be secured via the second end 110b of the resealing cap 100.

The first end 110a of the resealing cap 100 includes a sealing material 113. The sealing material 113 is puncturable. This will allow for a spike to be forced therethrough allowing access to the IV bag. In one embodiment the sealing material 113 is a thin membrane. In order to reseal the IV bag after the sealing material 113 has been punctured the resealing cap 100 may be changed.

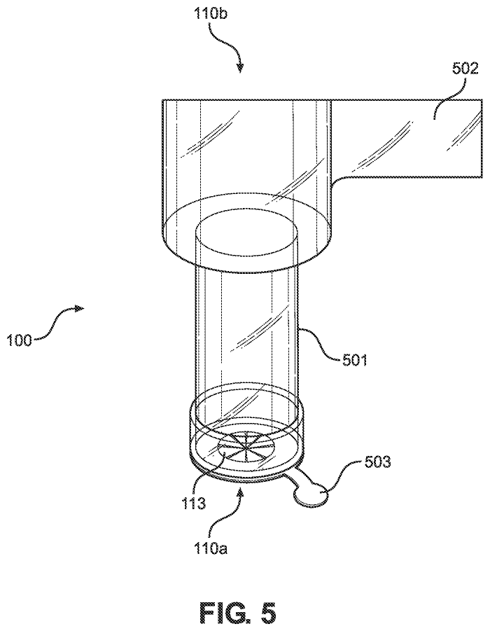

Referring now to FIG. 5, there is shown a perspective view of an embodiment of the resealing cap. In one embodiment the resealing cap 100 includes an extension tube 501. The extension tube 501 will allow for the sealing material 113 to be moved away from the original sealing material of the IV bag. This will ensure that there is a clean puncture of the resealing cap 100.

In one embodiment the resealing cap 100 includes an additional seal. Traditional IV bags include a pull tab ensuring the sealing material is not punctured until the desired time. Here in the interest of producing the most realistic training device, in some embodiments the resealing cap will include a pull tab 503. The pull tab 503 will cover the sealing material 113. In this embodiment the pull tab is removably secured to the resealing cap 100. In use the pull tab 503 may be removed prior to puncturing the sealing material 113.

In yet a further embodiment an additional tab 502 may be added to the device. The additional tab 502 will protrude from the exterior surface of the resealing cap 100. In one embodiment the tab 502 adds additional support for the application of the resealing cap 100. For example purposes, the tab 502 is shown located at the second end 110b of the resealing cap 100, however, one of ordinary skill in the art will understand that the tab 502 may be located in many places along the resealing cap 100 allowing for the same function. A user will be able to push against the tab 502 sliding the resealing cap 100 over the exit port of the IV bag. The same tab 502 can be used in the exact opposite manner in order to remove the resealing cap 100 after it has been used.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1) A resealing cap for an intravenous (IV) bag spike port, the resealing cap comprises:
a housing having a first end and a second end;
the housing having a passageway therethrough;
the first end of the housing is a closed end, wherein the material used to close the end is a puncturable material;
the second end of the housing is an open end.

2) The resealing cap for an intravenous (IV) bag spike port of claim 1, wherein the housing is made from a medical grade material.

3) The resealing cap for an intravenous (IV) bag spike port of claim 1, wherein the housing is made from an elastic material.

4) The resealing cap for an intravenous (IV) bag spike port of claim 1, wherein the second end of the housing has a removable seal placed about the open end.

5) The resealing cap for an intravenous (IV) bag spike port of claim 1, wherein the housing is cylindrical.

6) The resealing cap for an intravenous (IV) bag spike port of claim 5, further comprising a plurality of threads located around the interior of the housing.

7) The resealing cap for an intravenous (IV) bag spike port of claim 1, further comprising knurling about the exterior of the housing.

8) The resealing cap for an intravenous (IV) bag spike port of claim 1, further comprising a pull tab located at the second end of the resealing cap.

9) The resealing cap for an intravenous (IV) bag spike port of claim 1, further comprising an extension tube located between the first end and the second end of the resealing cap.

10) The resealing cap for an intravenous (IV) bag spike port of claim 1, further comprising a tab protruding from the resealing cap.
